(19) United States
(12) Patent Application Publication
(10) Pub. No.: US 2019/0345820 A1
(43) Pub. Date: Nov. 14, 2019

(54) SYSTEM AND METHOD OF SENSING HYDROCARBONS IN A SUBTERRANEAN ROCK FORMATION

(71) Applicant: General Electric Company, Schenctady, NY (US)

(72) Inventors: Dewey Lavonne Parkey, JR., Oklahoma City, OK (US); Yi Liao, Oklahoma City, OK (US); Stewart Brazil, Oklahoma City, OK (US); Richard Luht, Oklahoma City, OK (US)

(73) Assignee: General Electric Company, Schenctady, NY (US)

(21) Appl. No.: 16/474,854

(22) PCT Filed: Jan. 5, 2017
(86) PCT No.: PCT/CN2017/070276

§ 371 Date: Jun. 28, 2019

(51) Int. Cl. E21B 49/08; G01N 33/28; G01N 1/14

(52) U.S. Cl. CPC E21B 49/081; E21B 43/26; G01N 1/14; G01N 33/2823

(57) ABSTRACT

A method of sensing hydrocarbons in a subterranean rock formation, including advancing a drilling assembly within the subterranean rock formation. The drilling assembly is configured to discharge a first fluid into the subterranean rock formation, and wherein a second fluid flows past an exterior of the drilling assembly. The method further includes sampling at least one of the first fluid and the second fluid, thereby defining a sampled amount of fluid, and determining a hydrocarbon content of the sampled amount of fluid.

### BACKGROUND

The present disclosure relates generally to wellbore drilling and formation evaluation and, more specifically, to a Logging-While-Drilling or Measurement-While-Drilling sensing system for downhole hydrocarbon and gas species detection when forming a wellbore in a subterranean rock formation.

Hydraulic fracturing, commonly known as fracking, is a technique used to release petroleum, natural gas, and other hydrocarbon-based substances for extraction from underground reservoir rock formations, especially for unconventional reservoirs. The technique includes drilling a wellbore into the rock formations, and pumping a treatment fluid into the wellbore, which causes fractures to form in the rock formations and allows for the release of trapped substances produced from these subterranean natural reservoirs. At least some known unconventional subterranean wells are evenly fractured along the length of the wellbore. However, typically less than 50 percent of the fractures formed in the rock formations contribute to hydrocarbon extraction and production for the well. As such, hydrocarbon extraction from the well is limited, and significant cost and effort is expended for completing non-producing fractures in the wellbore.

### BRIEF DESCRIPTION

In one aspect, a method of sensing hydrocarbons in a subterranean rock formation is provided. The method includes advancing a drilling assembly within the subterranean rock formation, wherein the drilling assembly is configured to discharge a first fluid into the subterranean rock formation, and wherein a second fluid flows past an exterior of the drilling assembly. The method further includes sampling at least one of the first fluid and the second fluid, thereby defining a sampled amount of fluid, and determining a hydrocarbon content of the sampled amount of fluid.

In another aspect, a system for use in sensing hydrocarbons in a subterranean rock formation is provided. The system includes a drilling assembly configured to advance within the subterranean rock formation, and configured to discharge a first fluid into the subterranean rock formation. A second fluid flows past an exterior of the drilling assembly. The drilling assembly includes a fluid sampling mechanism configured to sample at least one of the first fluid and the second fluid, thereby defining a sampled amount of fluid, and at least one sensor configured to determine a hydrocarbon content of the sampled amount of fluid.

In yet another aspect, a sensing sub-assembly for use with a drilling assembly is provided. The sensing sub-assembly includes a cylindrical body including an internal flow channel extending therethrough, and the internal flow channel is configured to channel a first fluid therethrough. A sampling chamber is also defined therein, and the sampling chamber is coupled in flow communication with an ambient environment exterior of the cylindrical body. A second fluid flows within the ambient environment. The sensing sub-assembly further includes a fluid sampling mechanism configured to draw the second fluid into the sampling chamber, and at least one sensor coupled within the cylindrical body. The at least one sensor is configured to determine a hydrocarbon content of the second fluid within the sampling chamber.

### DRAWINGS

These and other features, aspects, and advantages of the present disclosure will become better understood when the following detailed description is read with reference to the accompanying drawings in which like characters represent like parts throughout the drawings, wherein:

FIG. 1 is a schematic illustration of an exemplary drilling assembly that may be used to form a wellbore;

FIG. 2 is a perspective view of an exemplary sensing sub-assembly that may be used in the drilling assembly shown in FIG. 1;

FIG. 3 is a cross-sectional view of the sensing sub-assembly shown in FIG. 2; and

FIG. 4 is an enlarged cross-sectional view of a portion of the sensing sub-assembly shown in FIG. 3.

Unless otherwise indicated, the drawings provided herein are meant to illustrate features of embodiments of the disclosure. These features are believed to be applicable in a wide variety of systems comprising one or more embodiments of the disclosure. As such, the drawings are not meant to include all conventional features known by those of ordinary skill in the art to be required for the practice of the embodiments disclosed herein.

### DETAILED DESCRIPTION

In the following specification and the claims, reference will be made to a number of terms, which shall be defined to have the following meanings.

The singular forms “a”, “an”, and “the” include plural references unless the context clearly dictates otherwise.

“Optional” or “optionally” means that the subsequently described event or circumstance may or may not occur, and that the description includes instances where the event occurs and instances where it does not.

Approximating language, as used herein throughout the specification and claims, may be applied to modify any quantitative representation that could permissibly vary without resulting in a change in the basic function to which it is related. Accordingly, a value modified by a term or terms, such as “about”, “approximately”, and “substantially”, are not to be limited to the precise value specified. In at least some instances, the approximating language may correspond to the precision of an instrument for measuring the value. Here and throughout the specification and claims, range limitations may be combined and/or interchanged. Such ranges are identified and include all the sub-ranges contained therein unless context or language indicates otherwise.

Embodiments of the present disclosure relate to a sensing system for downhole hydrocarbon and gas species detection when forming a wellbore in a subterranean rock formation. The sensing system is implemented as a standalone evaluation tool or installed as part of a wellbore drilling assembly. The sensing system obtains fluid samples from fluid flows that are either channeled into the wellbore through the drilling assembly or that backflow within the wellbore past the drilling assembly. More specifically, the samples are drawn into the drilling assembly for evaluation by at least one sensor, and the at least one sensor is likewise positioned within the drilling assembly to facilitate protecting the sensor from a caustic and abrasive wellbore environment. The sensor is used to determine the hydrocarbon content of the sampled fluid, and the results are used to identify potentially promising fracture initiation zones within the wellbore such that efficient and cost effective completion planning can be implemented.

For example, downhole hydrocarbon and gas species detection while drilling can identify zones of high permeability, such as open natural fractures, clusters of closed but unsealed natural fractures, larger pores and other formation features where hydrocarbons are stored. The analysis results can be used to identify the most promising fracture initiation points or zones, and the information can be used for completion planning, especially for unconventional reservoirs. In addition, the analysis results can be used to identify poor zones (no gas show), which facilitates reducing the time and effort of perforating and stimulating the poor zones. Another potential application is for geosteering assistance, wherein the real time gas show/species information is used to adjust the borehole position (e.g., inclination and azimuth angles) while drilling, such that a well having increased production can be formed. Finally, the sensing can also provide kick detection for real-time alerts of gas flow potential for safety and environmental considerations, thereby reducing the risk of system failure.

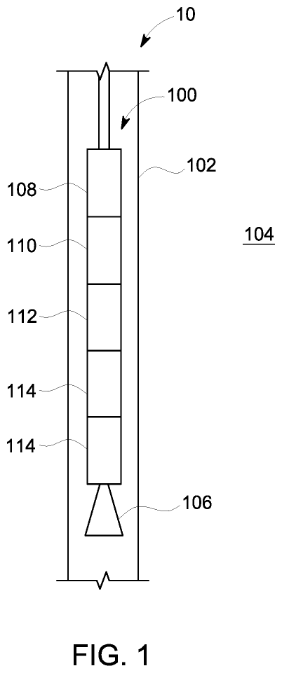

FIG. 1 is a schematic illustration of an exemplary hydraulic fracturing system 10. Hydraulic fracturing system 10 includes a drilling assembly 100 that may be used to form a wellbore 102 in a subterranean rock formation 104. In the exemplary embodiment, drilling assembly 100 includes a plurality of sub-assemblies and a drill bit 106. More specifically, the plurality of sub-assemblies include a measurement-while-drilling or logging-while-drilling sub-assembly 108, a sensing sub-assembly 110, a mud motor 112, and bent housing or rotary steerable system sub-assemblies 114 coupled together in series. Drilling assembly 100 includes any arrangement of sub-assemblies that enables drilling assembly 100 to function as described herein.

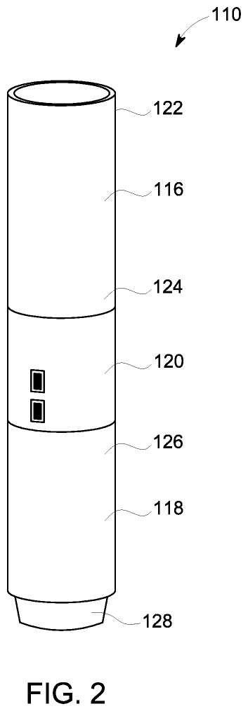

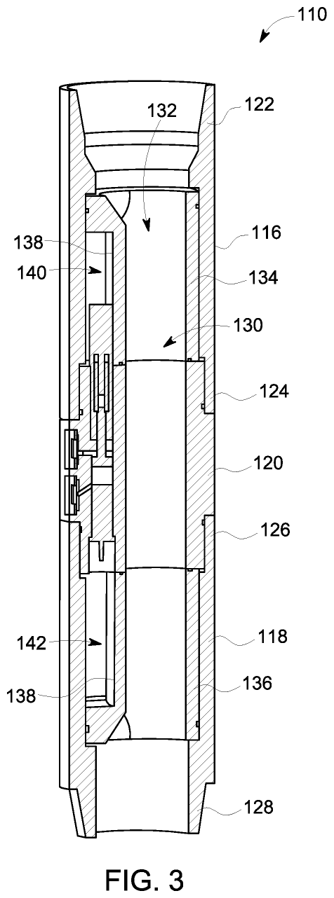

FIG. 2 is a perspective view of sensing sub-assembly 110 that may be used in drilling assembly 100 (shown in FIG. 1), and FIG. 3 is a cross-sectional view of sensing sub-assembly 110. In the exemplary embodiment, sensing sub-assembly 110 includes a first outer casing 116, a second outer casing 118, and a sampling hub 120 coupled therebetween. First outer casing 116 includes a first end 122 and a second end 124, and second outer casing 118 includes a first end 126 and a second end 128. First end 122, second end 124, first end 126, and second end 128 each include a threaded connection for coupling sensing sub-assembly 110 to one or more of the plurality of sub-assemblies of drilling assembly 100, and for coupling first outer casing 116 and second outer casing 118 to sampling hub 120.

Referring to FIG. 3, sensing sub-assembly 110 includes an interior 130 defined by an internal flow channel 132 extending therethrough. In addition, sensing sub-assembly 110 includes a first chassis 134 and a second chassis 136 coupled on opposing ends of sampling hub 120. Portions of internal flow channel 132 are defined by, and extend through, sampling hub 120, first chassis 134, and second chassis 136, as will be described in more detail below.

In the exemplary embodiment, first chassis 134 and second chassis 136 are each formed with a circumferential indent 138 such that a first electronics chamber 140 is defined between first chassis 134 and first outer casing 116, and such that a second electronics chamber 142 is defined between second chassis 136 and second outer casing 118. First electronics chamber 140 and second electronics chamber 142 are sealed from internal flow channel 132 such that electronics (not shown) housed therein are protected from high pressure fluid channeled through internal flow channel 132 during operation of drilling assembly 100.

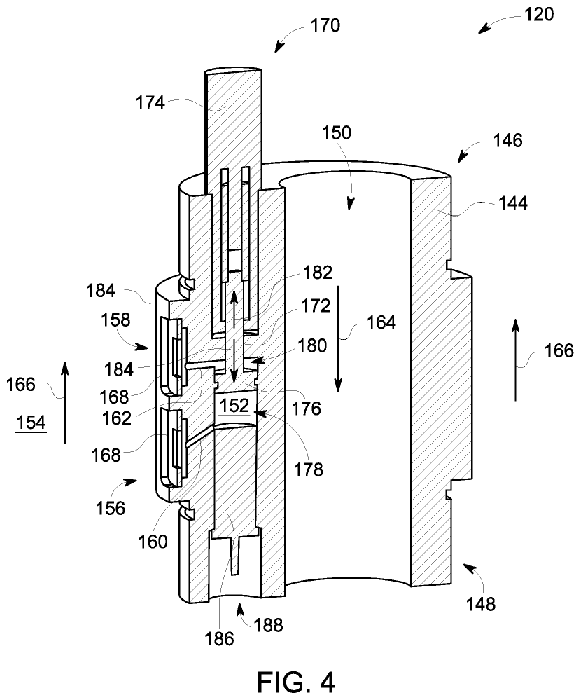

FIG. 4 is an enlarged cross-sectional view of a portion of sensing sub-assembly 110 (shown in FIG. 3). In the exemplary embodiment, sampling hub 120 includes a cylindrical body 144 including a first end 146 and a second end 148. First end 146 and second end 148 each include a threaded connection for coupling to first outer casing 116 and second outer casing 118 (both shown in FIG. 3), as described above. In addition, cylindrical body 144 includes an internal flow channel 150 extending therethrough that channels high pressure fluid during operation of drilling assembly 100, as will be described in more detail below.

Cylindrical body 144 further includes a sampling chamber 152 defined therein. Sampling chamber 152 is coupled in flow communication with an ambient environment 154 exterior of cylindrical body 144. More specifically, a first exterior flow opening 156 and a second exterior flow opening 158 are defined in cylindrical body 144. A first interior conduit 160 extends between sampling chamber 152 and first exterior flow opening 156, and a second interior conduit 162 extends between sampling chamber 152 and second exterior flow opening 158.

During operation of drilling assembly 100 (shown in FIG. 1), a first fluid 164 is pumped from surface equipment (not shown), is channeled through internal flow channels 132 and 150, and is discharged from drilling assembly 100, and a second fluid 166 backflows within wellbore 102 (shown in FIG. 1) past drilling assembly 100. First fluid 164 flows at a greater pressure than second fluid 166, and second fluid 000 includes a portion of first fluid 000 and constituents of subterranean rock formation 104. In one embodiment, as will be explained in more detail below, second fluid 166 is selectively channeled into sampling chamber 152 through first exterior flow opening 156 and first interior conduit 160. Sampling hub 120 further includes a filter 168 that covers first exterior flow opening 156 and second exterior flow opening 158 such that particulate matter entrained in second fluid 166 is restricted from entering sampling chamber 152.

In the exemplary embodiment, sensing sub-assembly 110 includes a fluid sampling mechanism 170 coupled within cylindrical body 144. Fluid sampling mechanism 170 includes a piston 172 and an actuating device 174 that controls operation of piston 172. Actuating device 174 is any device capable of causing piston 172 to move. An exemplary actuating device 174 includes, but is not limited to, an electric motor.

Piston 172 includes a piston head 176 that is positioned within sampling chamber 152. Piston 172 is selectively translatable within sampling chamber 152 to facilitate drawing second fluid 166 into sampling chamber 152. More specifically, piston head 176 is positioned within sampling chamber 152 such that sampling chamber 152 is partitioned into a first portion 178 and a second portion 180. First portion 178 is coupled in flow communication with first interior conduit 160, and second portion is coupled in flow communication with second interior conduit 162.

In operation, actuating device 174 causes piston 172 to translate in a first direction 182 such that second fluid 166 is drawn into sampling chamber 152. More specifically, translating piston 172 in first direction 182 facilitates forming a negative pressure within sampling chamber 152. As such, a sampled amount of second fluid 166 is drawn through first exterior flow opening 156 and into first portion 178 of sampling chamber 152. As will be described in more detail below, measurements of the sampled amount of second fluid 166 within first portion 178 of sampling chamber 152 are taken before being discharged back into ambient environment 154. More specifically, actuating device 174 causes piston 172 to translate in a second direction 184 such that the sampled amount of second fluid 166 is discharged from first exterior flow opening 156. In addition, second exterior flow opening 158 and second interior conduit 162 couple second portion 180 of sampling chamber 152 in flow communication with ambient environment 154. As such, second exterior flow opening 158 and second interior conduit 162 provide a pressure relief flow channel to facilitate enabling translation of piston 172 within sampling chamber 152.

In the exemplary embodiment, sensing sub-assembly 110 further includes at least one sensor 186 coupled within cylindrical body 144. More specifically, cylindrical body 144 further includes a sensor chamber 188 defined therein, and sensor 186 is positioned within sensor chamber 188. Sensor chamber 188 is positioned adjacent to sampling chamber 152 such that sensor 186 is capable of analyzing the fluid contained therein. For example, as described above, sensor 186 determines a hydrocarbon content of the sampled amount of fluid contained within sampling chamber 152. Exemplary sensors include, but are not limited to, an acoustic sensor, a nuclear magnetic resonance sensor, an electrical impedance spectroscopy sensor, and an optical spectroscopy sensor. Alternatively, any sensors for determining the hydrocarbon content of the fluid contained within sampling chamber 152 may be utilized that enables sensing sub-assembly 110 to function as described herein.

In an alternative embodiment, sensing sub-assembly 110 is further capable of sampling and analyzing first fluid 164 channeled through internal flow channel 150. More specifically, in the alternative embodiment, first interior conduit 160 is sealable, and a third interior conduit (not shown) extends between internal flow channel 150 and sampling chamber 152. In a further alternative embodiment, more than one sampling chamber and fluid sampling mechanism setup is included within sensing sub-assembly 110, wherein a first setup samples and analyzes first fluid 164 and a second setup samples and analyzes second fluid 166.

A method of operating hydraulic fracturing system 10 (shown in FIG. 1) is also described herein. The method includes advancing drilling assembly 100 within subterranean rock formation 104. Drilling assembly 100 discharges first fluid 164 into subterranean rock formation 104, and second fluid 166 flows past an exterior of drilling assembly 100. The method further includes sampling at least one of first fluid 164 and second fluid 166, thereby defining a sampled amount of fluid, and determining a hydrocarbon content of the sampled amount of fluid.

As described above, the results are used to identify potentially promising fracture initiation zones within the wellbore such that efficient and cost effective completion planning can be implemented. For example, the method further includes identifying fracture initiation locations within subterranean rock formation 104 based on the hydrocarbon content of the sampled amount of fluid. For example, in one embodiment, multiple samples of at least one of first fluid 164 and second fluid 166 are obtained at different locations within subterranean rock formation 104 as drilling assembly 100 advances within subterranean rock formation 104. The hydrocarbon content of the sampled fluid is determined at the different locations within the subterranean rock formation, and the data is analyzed to identify fracture initiation locations.

For example, in one embodiment, identifying fracture initiation locations includes determining a hydrocarbon content of a sampled amount of second fluid 166, wherein the fracture initiation locations are identified when the hydrocarbon content of the sampled amount of second fluid 166 is greater than a predetermined threshold. Moreover, in one embodiment, identifying fracture initiation locations includes determining a hydrocarbon content of a sampled amount of first fluid 164 when drilling assembly 100 is at a location within subterranean rock formation 104, determining a hydrocarbon content of a sampled amount of second fluid 166 when drilling assembly 100 is at the location, and determining when a difference in the hydrocarbon content of the sampled first fluid 164 when compared to the hydrocarbon content of the sampled second fluid 166 is greater than a predetermined threshold. For example, first fluid 164 is substantially hydrocarbon free, and thus the hydrocarbon content of the sampled first fluid 164 provides a baseline value in which to compare to the hydrocarbon content of the sampled second fluid 166.

Moreover, in the exemplary embodiment, the hydrocarbon content data is either stored within drilling assembly 100 for later accessibility, or transmitted to a surface site (not shown) located above subterranean rock formation 104. The hydrocarbon content data is logged and analyzed for later use in identifying potentially promising fracture initiation zones, as described above. For example, in an alternative embodiment, the fracture initiation zones are identified by tracking relative changes in the hydrocarbon content at different locations within wellbore 102, and determining the location within subterranean rock formation 104 where the hydrocarbon content increases dramatically when compared to other locations in subterranean rock formation 104.

In some embodiments, the method further includes alternatingly sampling first fluid 164 and second fluid 166 as drilling assembly 100 advances within subterranean rock formation 104, and modifying a trajectory of drilling assembly 100 based on the hydrocarbon content of the sampled amount of fluid.

The systems and assemblies described herein facilitate providing at least semi-continuous hydrocarbon and gas species detection feedback when drilling unconventional subterranean wells. More specifically, the drilling assembly facilitates sampling and analyzing fluid used in the drilling process in a fast and efficient manner. The data obtained from the analysis of the fluid samples can then be used to determine zones within a wellbore that have either a low likelihood or a high likelihood of having a high hydrocarbon content. As such, the zones having a high hydrocarbon content are identified, and fracture completion planning resulting in improved well production is determined.

An exemplary technical effect of the systems and assemblies described herein includes at least one of: (a) providing real-time and continuous hydrocarbon and gas species detection feedback when forming a well in a subterranean rock formation; (b) identifying potentially promising fracture initiation zones within a wellbore; (c) improving hydrocarbon production for wells; (d) providing geosteering assistance for the drilling assembly; and (e) providing kick detection for real-time gas flow potential safety alerts.

Exemplary embodiments of a drilling assembly and related components are described above in detail. The drilling assembly is not limited to the specific embodiments described herein, but rather, components of systems and/or steps of the methods may be utilized independently and separately from other components and/or steps described herein. For example, the configuration of components described herein may also be used in combination with other processes, and is not limited to practice with only drilling and sensing assemblies and related methods as described herein. Rather, the exemplary embodiment can be implemented and utilized in connection with many applications where sampling and analyzing one or more fluids is desired.

Although specific features of various embodiments of the present disclosure may be shown in some drawings and not in others, this is for convenience only. In accordance with the principles of embodiments of the present disclosure, any feature of a drawing may be referenced and/or claimed in combination with any feature of any other drawing.

This written description uses examples to disclose the embodiments of the present disclosure, including the best mode, and also to enable any person skilled in the art to practice embodiments of the present disclosure, including making and using any devices or systems and performing any incorporated methods. The patentable scope of the embodiments described herein is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A method of sensing hydrocarbons in a subterranean rock formation, said method comprising:
advancing a drilling assembly within the subterranean rock formation, wherein the drilling assembly is configured to discharge a first fluid into the subterranean rock formation, and wherein a second fluid flows past an exterior of the drilling assembly;
sampling at least one of the first fluid and the second fluid, thereby defining a sampled amount of fluid; and
determining a hydrocarbon content of the sampled amount of fluid.

2. The method in accordance with claim 1 further comprising identifying potential fracture initiation locations within the subterranean rock formation based on the hydrocarbon content of the sampled amount of fluid.

3. The method in accordance with claim 2, wherein identifying fracture initiation locations comprises determining a hydrocarbon content of a sampled amount of the second fluid, wherein the fracture initiation locations are identified when the hydrocarbon content of the sampled amount of the second fluid is greater than a predetermined threshold.

4. The method in accordance with claim 2, wherein identifying fracture initiation locations comprises:
determining a hydrocarbon content of a sampled amount of the first fluid;
determining a hydrocarbon content of a sampled amount of the second fluid; and
determining when a difference in the hydrocarbon content of the sampled amount of the first fluid when compared to the hydrocarbon content of the sampled amount of the second fluid is greater than a predetermined threshold.

5. The method in accordance with claim 1, wherein sampling at least one of the first fluid and the second fluid comprises sampling additional amounts of at least one of the first fluid and the second fluid at different locations within the subterranean rock formation as the drilling assembly advances within the subterranean rock formation.

6. The method in accordance with claim 1, wherein sampling at least one of the first fluid and the second fluid comprises alternatingly sampling the first fluid and the second fluid as the drilling assembly advances within the subterranean rock formation.

7. The method in accordance with claim 1, wherein advancing a drilling assembly comprises modifying a trajectory of the drilling assembly based on the hydrocarbon content of the sampled amount of fluid.

8. A system for use in sensing hydrocarbons in a subterranean rock formation, said system comprising:
a drilling assembly configured to advance within the subterranean rock formation, and configured to discharge a first fluid into the subterranean rock formation, wherein a second fluid flows past an exterior of the drilling assembly, said drilling assembly comprising:
a fluid sampling mechanism configured to sample at least one of the first fluid and the second fluid, thereby defining a sampled amount of fluid; and
at least one sensor configured to determine a hydrocarbon content of the sampled amount of fluid.

9. The system in accordance with claim 8, wherein said at least one sensor comprises at least one of an acoustic sensor, a nuclear magnetic resonance sensor, an electrical impedance spectroscopy sensor, and an optical spectroscopy sensor.

10. The system in accordance with claim 8, wherein said at least one sensor is configured to determine a hydrocarbon content of a sampled amount of the second fluid, wherein potential fracture initiation locations in the subterranean rock formation are identified when the hydrocarbon content of the sampled amount of the second fluid is greater than a predetermined threshold.

11. The system in accordance with claim 8, wherein said at least one sensor is configured to:
determine a hydrocarbon content of a sampled amount of the first fluid; and
determine a hydrocarbon content of a sampled amount of the second fluid, wherein fracture initiation locations in the subterranean rock formation are identified when a difference in the hydrocarbon content of the sampled amount of the first fluid when compared to the hydrocarbon content of the sampled amount of the second fluid is greater than a predetermined threshold.

12. The system in accordance with claim 8, wherein said fluid sampling mechanism is configured to sample additional amounts of at least one of the first fluid and the second fluid at different locations within the subterranean rock formation as the drilling assembly advances within the subterranean rock formation.

13. The system in accordance with claim 8, wherein said drilling assembly comprises a sensing sub-assembly comprising:
a cylindrical body that comprises:
an internal flow channel extending therethrough, said internal flow channel configured to channel the first fluid therethrough; and
a sampling chamber defined therein, wherein said fluid sampling mechanism is configured to draw at least one of the first fluid and the second fluid into said sampling chamber.

14. The system in accordance with claim 13, wherein said fluid sampling mechanism is configured to alternatingly sample the first fluid and the second fluid within said sampling chamber.

15. The system in accordance with claim 13, wherein said fluid sampling mechanism comprises a piston selectively translatable within said sampling chamber.

16. A sensing sub-assembly for use with a drilling assembly, said sensing sub-assembly comprising:
a cylindrical body comprising:
an internal flow channel extending therethrough, said internal flow channel configured to channel a first fluid therethrough; and
a sampling chamber defined therein, said sampling chamber coupled in flow communication with an ambient environment exterior of said cylindrical body, wherein a second fluid flows within the ambient environment;
a fluid sampling mechanism configured to draw the second fluid into said sampling chamber; and
at least one sensor coupled within said cylindrical body, said at least one sensor configured to determine a hydrocarbon content of the second fluid within said sampling chamber.

17. The sensing sub-assembly in accordance with claim 16, wherein said fluid sampling mechanism is configured to draw the first fluid into said sampling chamber when not filled with the second fluid, and said at least one sensor is configured to determine a hydrocarbon content of the first fluid within said sampling chamber.

18. The sensing sub-assembly in accordance with claim 17, wherein said fluid sampling mechanism is configured to alternatingly sample the first fluid and the second fluid within said sampling chamber.

19. The sensing sub-assembly in accordance with claim 16, wherein said at least one sensor comprises at least one of an acoustic sensor, a nuclear magnetic resonance sensor, an electrical impedance spectroscopy sensor, and an optical spectroscopy sensor.

20. The sensing sub-assembly in accordance with claim 16, wherein said fluid sampling mechanism comprises a piston selectively translatable within said sampling chamber.
